(19) United States
(12) Patent Application Publication
(10) Pub. No.: US 2018/0022081 A1
(43) Pub. Date: Jan. 25, 2018

(54) Aesthetic improvements in hearing protection

(71) Applicant: Brian Felsen, Minnetonka, MN (US)

(72) Inventor: Brian Felsen, Minnetonka, MN (US)

(21) Appl. No.: 15/657,865

(22) Filed: Jul. 24, 2017

Related U.S. Application Data
(60) Provisional application No. 62/365,590, filed on Jul. 22, 2016.

(51) Int. Cl. B41F 17/28; A61F 11/08; A61F 11/14

(52) U.S. Cl. CPC B41F 17/28; A61F 11/14; A61F 11/08

(57) ABSTRACT

Methods for application of designs to devices such as headphones, earmuffs, and/or earplugs. At least one exterior surface of the device is painted, and a printed film is placed in a water tank at the water surface for separating the image from the film, such that the image is transferred to the exterior surface of the device. The device is rinsed and dried, and top coat applied. Alternatively, earplugs may have designs applied thereto utilizing a custom fixture configured to hold one or a plurality of devices concurrently with the exterior surface for decoration facing outwardly or upwardly and then transferring an image to the exterior surface using an indirect offset decorating process.

### CROSS-REFERENCE TO RELATED APPLICATION

This application claims the benefit of U.S. provisional application Ser. No. 62/365,590, filed Jul. 22, 2016, the contents of which are hereby incorporated in their entirety.

### BACKGROUND OF THE INVENTION

Proper hearing protection equipment includes earmuffs, headphones and ear plugs. It is important to protect the hearing of infants, children and adults to prevent eardrum rupture, temporary, long term and/or permanent hearing loss.

Approximately 50 million Americans are affected by Tinnitus (ringing or buzzing in the ears) and on a daily basis as many as 22 million people are exposed to hazardous sounds levels at work.

Most hearing protection items are industrial in design and appearance or lack individualization.

### SUMMARY

One aspect of the present disclosure relates to a method of applying a selected design or graphic to at least one exterior surface of a device worn and covering at least one ear. The device may be a headphone or an earmuff and may include devices intended for protection of hearing or transmission of sound. The method comprises painting at least one exterior surface of the device in one or more pre-selected colors to provide a base coat to the exterior surface and printing a film with the selected design or graphic, wherein the film comprises a polyvinyl alcohol cubic printing film. The method further comprises selectively placing the printed film in a water tank at the water surface for separating the image from the film and transferring the selected design or graphic to the exterior surface of the device. Next, the method comprises rinsing away film residue and adhering ink of the design or graphic to the surface and allowing the exterior surface to dry and coating a final protective coating, top coat or sealant on exterior surface.

Another aspect of the present disclosure relates to a method of applying a selected design or graphic to at least one exterior surface of a device for hearing protection, where the method comprises creating a custom fixture to adequately hold, imprint, and mass produce devices with at least one decorated exterior surface. The method comprises utilizing the custom fixture which is configured to hold one or a plurality of devices concurrently with the exterior surface for decoration facing outwardly or upwardly and transferring an image to the exterior surface using an indirect offset decorating process via a cliché and silicone transfer pad.

### BRIEF DESCRIPTION OF THE DRAWINGS

FIG. 1 illustrates a method of applying colors and a selected image to a hearing protection device.

FIG. 2 illustrates hearing protection devices before (left) and after (right) decoration by a process described herein and applied to said device.

FIG. 3 illustrates hearing protection devices before (left) and after (right) decoration by a process described herein and applied to said device.

FIG. 4 is a top view of a custom fixture of the present disclosure.

FIG. 5 is a cross-sectional view of the custom fixture about line A-A.

FIG. 6 is a top view of an additional configuration of the custom fixture.

FIG. 7 illustrates a method of applying an image to a hearing protection device.

FIG. 8 is a top view of the earplugs decorated according to the present disclosure.

### DETAILED DESCRIPTION

The present disclosure relates to decorated infant, youth, and adult hearing protection devices. These devices may include earmuffs, headphones, earplugs or other similar devices. Headphones and ear-buds are also subject to this disclosure. The terms earmuffs/headphone are used interchangeably throughout the disclosure, hearing protection devices as used herein can include devices for covering ears as well as listening devices, such as earmuffs/headphones/earbuds having bluetooth compatibility to stream music and entertainment and also includes noise-limiting technology earbuds and headphones that do not output over a safe decibel level. Devices include devices that cover ears or are inserted into the ear canal and for which are decorated by the application of printed graphics to exterior surfaces thereof.

Components, such as external surfaces (e.g. hard, non-porous outer shell of earmuffs/headphones) go through a painting process that prepares the surface of the earmuff component. A selected paint color or colors can be used when applicable by graphic as an under coat or base color. Then a printing film, such as a polyvinyl alcohol cubic printing film that has been printed with the selected graphic or design to be decorated on the earmuff component is carefully placed on the surface of a decorating water tank. In the water tank, this dissolves the medium but not the graphic or design, and allows the design to transfer to the surface of the earmuff or headphone etc. Thereafter, once the component is placed into the pattern and residue is rinsed away thoroughly, the ink has adhered to the earmuff component and will not wash off. It is at this time that the component is allowed to dry before a final protective coating, top coat or sealant is adhered to the surface.

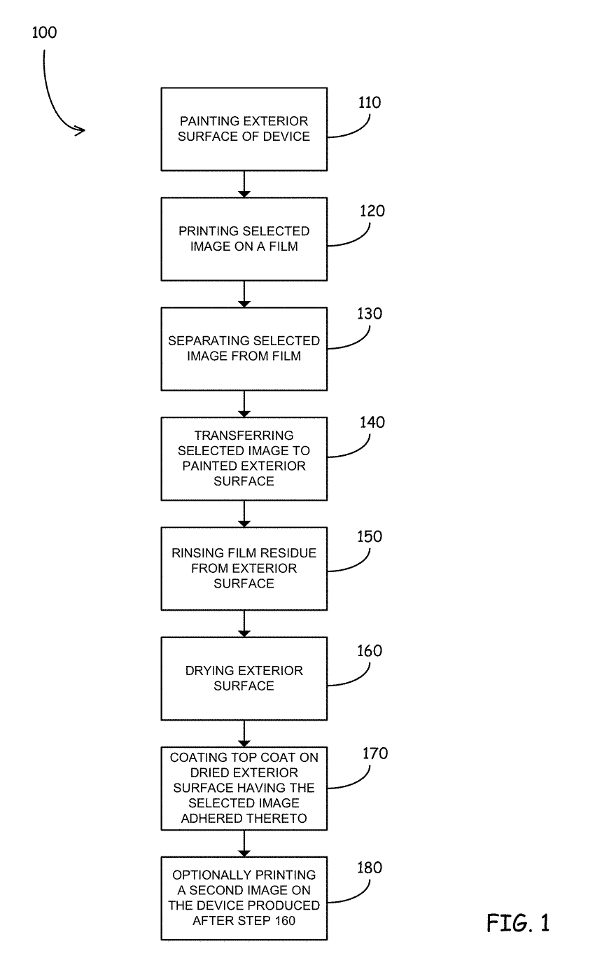

In further detail, the hard or non-porous exterior surfaces of headphones or earmuffs can be selectively decorated with graphics by a process such as hydrodipping as described in FIG. 1. The process comprises hydrodipping the selected exterior surface of the earmuffs or headphones which includes graphic transfer in water, the transfer of high definition image patterns to the outer earmuff surface. The process is carried out by systematically lowering the components into a hydrodipping tank full of continuously warmed water (e.g. 90 degrees F.) on/at its surface such that upon the dissolution of the decorated film, the selected image remains in its original representation but separated from the film such that the image is transferred, permanently, to the earmuff and/or headphone surface.

Earmuff components may painted colors strategically for contrasting backgrounds or other reasons, which may be pre-determined by the final design of each style coating. Once the earmuff component is completely dry after initial painting of the base coat, and the selected film is prepared, the earmuff components are ready for the Hydrodipping decoration process. Approximately 5-10 seconds after spraying an activator composition across the surface of the film, the earmuff component is smoothly, at an angle of about 35-40 degrees, lowered directly though the film and image (the image positioned on the surface of the water) into the water. Once the earmuff component(s) are completely submerged into the water, the component(s) is/are held vertically and shook up and down several times to separate the film residues from the surface of the film, leaving the ink on the component in the selected design.

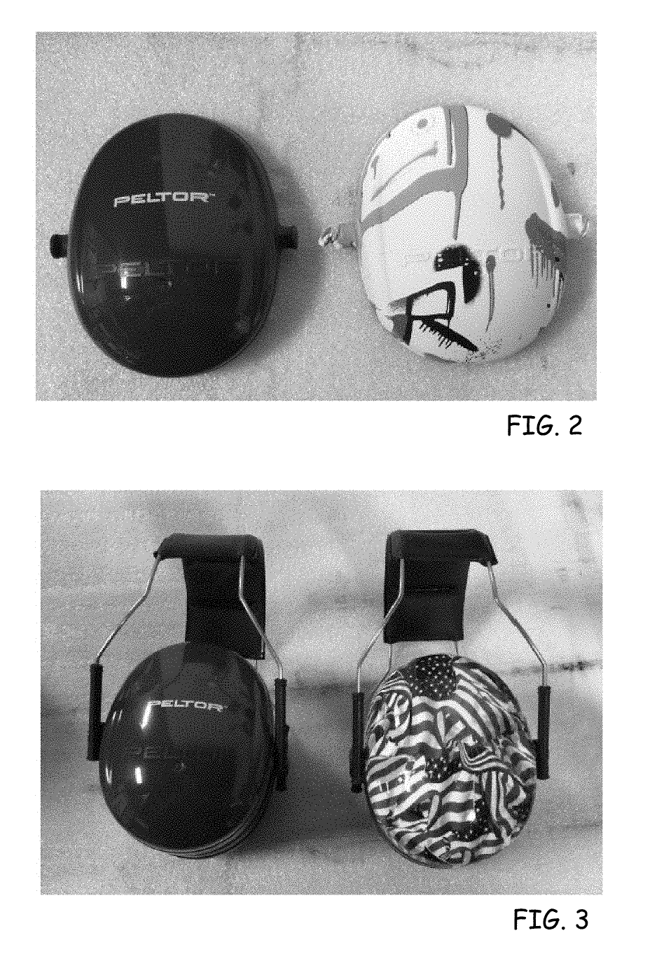

Once the components have gone through the graphic transfer process they are ready for power-washing and the earmuff components are thoroughly dried. Thereafter, the component is sprayed with any one of a protective coating, such as a high-gloss or a satin-matte finishes, a glow in the dark finish, and/or others specific to the design and specifications of each application. Examples of the devices before and after undergoing the method described in FIG. 1 are illustrated in FIGS. 2-3.

Referring back to FIG. 1, the method 100 of applying a selected design or graphic to at least one exterior surface of a device worn and covering at least one ear generally comprises (110) painting the at least one exterior surface of the device; (120) printing a film with the selected design or graphic; (130) separating the selected design or graphic image from the film; and (140) transferring the selected design or graphic to the exterior surface of the device. The method also comprises (150) rinsing away film residue and adhering ink of the design or graphic from the exterior surface; (160) allowing the exterior surface to dry; and (170) coating a final protective coating, top coat or sealant on exterior surface. The method 100 may optionally further comprise (180) printing a second selected graphic or design on at least a portion of the printed exterior surface wherein the selected graphic or design is a logo or insignia.

Once decorated, the components (exterior surface coverings for ear protection) are assembled into the hearing protection device and packaged for retail and promotional purposes. In one embodiment, an imprinted logo may also be placed strategically on the earmuff component prior to the coating process is selected. Such additional imprinting may comprise logos, insignias or monograms which are printed on top of the decorated surface pattern. The additional or second image would be printed on top of the hydrodipped component using a process which utilizes a custom fixture for printing a logo on earmuff or headphone components and connected to a machine for offset decorating process via a cliché and silicone transfer pad, where the custom fixture is fabricated in the shape of an earmuff component. This method of second printing may be done by a method similar to the method for printing ends of foam ear plugs as discussed further below.

The method of printing the decorations on the components allows for “color matching” of the components, meaning that the external surface of the components may be printed with one or more colors selected and matching the colors associated with a company's logo or a sports team or college's team colors, etc. The method allows for multi and/or full color applications on the components.

The earmuff component may alternatively be decorated using a direct jet UV printer, wherein the multi-colored images are printed directly on the ear-muff components.

Another aspect of the present disclosure relates to decorated end caps of earplugs such as foam earplugs. By creating a custom fixture 200 to adequately hold, imprint, and mass produce for retail and promotional product purposes. This is accomplished by utilizing a custom fixture 200 built specifically to hold one or multiple foam earplugs at one time with the end cap facing up so that the image is transferred to the earplug using an indirect offset decorating process via a cliché and silicone transfer pad.

The custom fixture 200 is configured for insertion into a machine for transfer and includes fasteners in the corners of the fixture for securing the custom fixture 200 in place for the decorating process.

With respect to the earplugs, specifically foam based earplugs, in general, the shape of the earplug includes one end that is rounded for insertion into the ear canal and the other (opposing) end has a generally flat surface that faces away from the ear and extends slightly from the ear canal when worn properly. It is this flat end that is decorated or customized so that it is visible to people when they look over at an individual's auricle “pinna” (the largely cartilaginous projecting portion of the external ear). Some earplug styles also comprise two flat sides (instead of the rounded insertion end), in this embodiment, both ends (flat) can be decorated for interchangeable use.

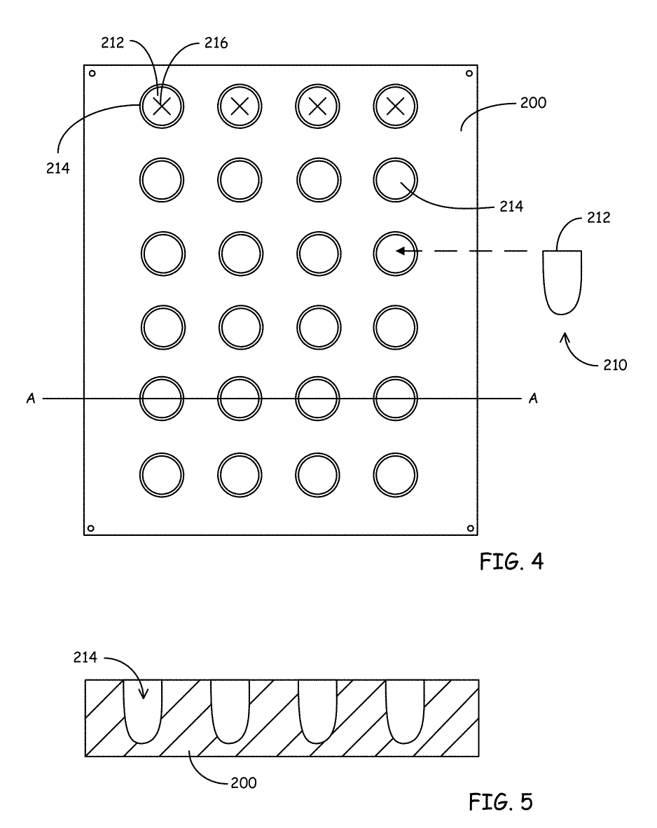

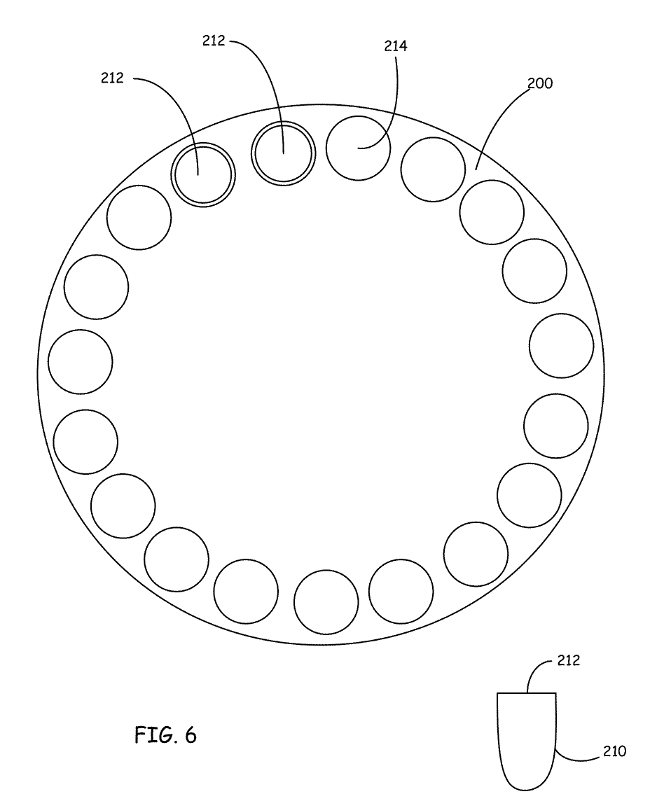

Referring to FIGS. 4-6, the custom fixture 200 can be used to hold a single ear plug 210 for decorating or to hold a plurality of ear plugs 210 for decorating at the same time. The end cap 212 or flat surface of the ear plug(s) 210 is/are exposed in the custom fixture 200 as the earplug 210 is inserted into a corresponding aperture 214 in the fixture 200 in the same direction as the earplug 210 would be inserted into a user's ear. The imprint area 216 is an area corresponding to the end cap 212 or generally flat surface of the earplug 210 for imprinting. The custom fixture 200 is configured to hold one or more earplugs and insertable into a machine for transferring the design to the earplug. As illustrated in FIGS. 4 and 6, the custom insert 200 may be of any geometric shape having one or more apertures 214 having a floor or otherwise configured to receive and hold an earplug 201 inserted therein in an upright position for imprinting the exposed imprint area 216 of the end cap 212.

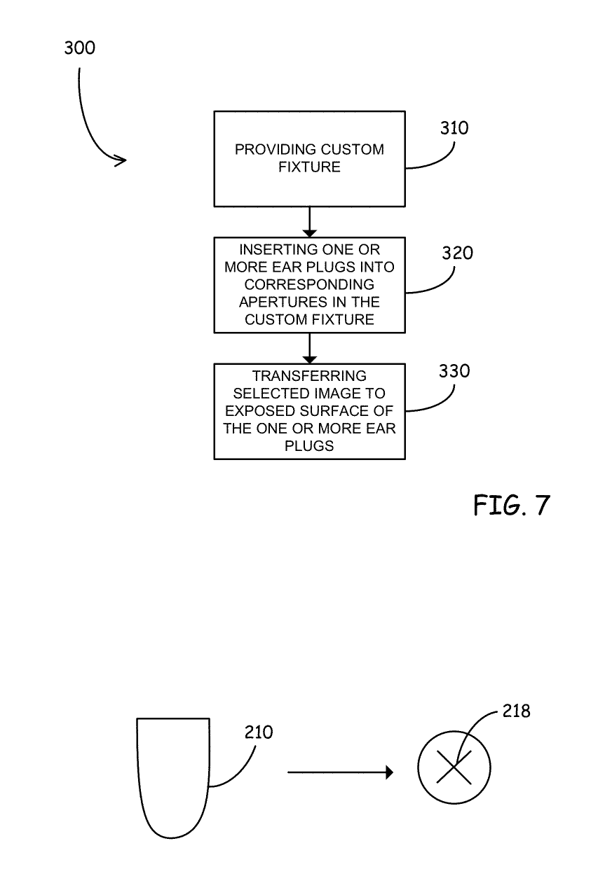

Referring to FIGS. 7-8, the method 300 of decorating the earplugs 210 comprises (310) providing the custom fixture 200 to adequately hold, imprint, and mass produce devices with at least one decorated exterior surface and (320) inserting one or more earplugs 210 into corresponding apertures 214 in the custom fixture 200 that are each configured to receive one earplug 210. The method 300 then comprises (330) transferring the selected image 218 to the exposed surface 212 of the one or more earplugs 210. The decorating process may be an indirect offset decorating process via a cliché and silicone transfer pad.

It is also contemplated that in addition to protective coverings for ears, the processes herein applied to earmuffs can also be applied to and used to personalize headphones and/or earbuds such as those for delivering sound (e.g. headphones for listening to music or other audio casts, rather than protecting hearing from loud or prolonged sounds).

The products are products designed to reduce the incidence and prevalence of recreational, occupational, music, and noise-induced hearing loss (NIHL) and Tinnitus (ringing of the ear, which is an early indicator of hearing loss) in a fun and fashionable way. The processes herein allow for the customization and decoration of personal protective equipment in the form of ear and hearing protection devices which may further encourage youth as well as people of all ages to wear the proper protection to protect their hearing.

Although the present disclosure has been described with reference to preferred embodiments, workers skilled in the art will recognize that changes may be made in form and detail without departing from the spirit and scope of the disclosure.

1. A method of applying a selected design or graphic to at least one exterior surface of a device worn and covering at least one ear, the method comprising:
painting the at least one exterior surface of the device in one or more pre-selected colors to provide a base coat to the exterior surface;
printing a film with the selected design or graphic;
selectively placing the printed film in a water tank at the water surface for separating the image from the film;
transferring the selected design or graphic to the exterior surface of the device;
rinsing away film residue and adhering ink of the design or graphic to the surface;
allowing the exterior surface to dry; and
coating a final protective coating, top coat or sealant on exterior surface.

2. The method of claim 1, wherein the film comprises a polyvinyl alcohol cubic printing film.

3. The method of claim 1, wherein the device is an earmuff or headphone.

4. The method of claim 1, wherein the device is designed for hearing protection.

5. The method of claim 1 wherein the method is a hydropdipping process.

6. The method of claim 5, and further comprising transferring a second selected image to at least a portion of the exterior surface using an indirect offset decorating process via a cliché and silicone transfer pad.

7. The method of claim 1, and further comprising printing a second selected graphic or design on at least a portion of the printed exterior surface wherein the selected graphic or design is a logo or insignia.

8. A method of applying a selected design or graphic to at least one exterior surface of a device for hearing protection, the method comprising:
providing a custom fixture to adequately hold, imprint, and mass produce devices with at least one decorated exterior surface;
utilizing the custom fixture which is configured to hold one or a plurality of devices concurrently with the exterior surface for decoration facing outwardly or upwardly;
transferring an image to the exterior surface using an indirect offset decorating process via a cliché and silicone transfer pad.

9. The method of claim 8, wherein the device is a foam earplug and one or both ends of the foam earplug are decorated.

9. The method of claim 8, wherein the custom fixture is configured to hold up to eighteen devices at once for concurrently applying the selected design to the at least one exterior surface of a plurality of devices for hearing protection.

10. The method of claim 9, wherein the custom fixture is circular in shape.

11. The method of claim 9, wherein the custom fixture is square or rectangular in shape.
